(19) United States
(12) Patent Application Publication
(10) Pub. No.: US 2010/0143439 A1
(43) Pub. Date: Jun. 10, 2010

(54) Hybrid Biomimetic Particles, Methods of Making Same and Uses Therefor

(75) Inventors: A. Champa Jayasuriya, Sylvania, OH (US); Nabil A. Ebraheim, Toledo, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: UNIVERSITY OF TOLEDO, Toledo, OH (US)

(21) Appl. No.: 12/596,221

(22) PCT Filed: Apr. 14, 2008
(86) PCT No.: PCT/US08/04825

§ 371 Date: Nov. 4, 2009

Related U.S. Application Data
(60) Provisional application No. 60/923,715, filed on Apr. 16, 2007.

(51) Int. Cl. A61K 35/12; A61F 2/00; A61K 9/10; A61K 38/18; A61P 19/08; A61P 19/04

(52) U.S. Cl. 424/423; 424/484; 424/488; 424/93.7; 514/12

(57) ABSTRACT

Biomimetic microparticles for generation of tissue includes a biodegradable tissue replacement/repair matrix formed of a cross-linked scaffold material using a non-toxic multi-valent ionic cross-linking agent. One or more bioactive materials can be encapsulated and/or coated onto the biodegradable tissue replacement/repair matrix. The biomimetic microparticles are formed without exposure to high temperatures, high pressures, high voltages, and toxic chemicals.

### CROSS-REFERENCE TO RELATED APPLICATIONS AND STATEMENT REGARDING SPONSORED RESEARCH

The present invention claims the benefit of the provisional patent application Ser. No. 60/923,715 filed Apr. 16, 2007. This invention was not made with any government support under and the government has no rights in this invention.

### BACKGROUND OF THE INVENTION

Over 600,000 bone grafting procedures are performed annually in the United

States. These numbers will grow as the life expectancy of the population increases. The estimated cost of these procedures approaches $2.5 billion per year. The two main types of bone grafts currently used are autografts and allografts.

An autograft is a section of bone taken from the patient's own body, whereas an allograft is taken from a cadaver. These types of grafts are limited due to some uncontrollable factors. For autografts, the key limitation is donor site morbidity, in which the remaining tissue at the harvest site is damaged by the removal of the graft resulting in surgical scars, blood loss, pain, prolonged surgical time and rehabilitation time, increased exposure to blood products, and infection risk. For allografts, key limitations are unfavorable immunologic response and transmission of viral diseases.

Tissue engineering is an emerging interdisciplinary field that seeks to apply the principles of biology and engineering to the development of viable tissue substitutes that are capable of restoring and maintaining the function of normal human tissues. An ideal bone substitute would possess the biological advantages of an autograft and supply advantages of an allograft, but alleviate the complications of each of these types of grafts.

Considering these issues it is necessary to develop bone substitute materials that can apply for bone healing without any problems.

Bone formation is directed by the coordinated expression of many molecules, including mitogenic growth factors, bone morphogenetic proteins and specific transcription factors, which utilize developmentally derived signals to induce cellular and molecular stimuli to guide cellular commitment and differentiation in the proper spatial and temporal sequence. Growth factors describe a group of soluble proteins that act as signaling agents for particular cell types and influence critical functions, such as cell division, matrix synthesis and tissue differentiation, by receptor-ligand binding. In addition, growth factors play many important roles in bone formation and bone repair. For example, a number of growth factors and their downstream molecular targets have been characterized during osteoblast differentiation.

Bone morphogenetic proteins are secreted signaling molecules that have a variety of functions during development and cell differentiation. They were identified due to their remarkable ability to induce cartilage and bone formation from non-skeletal mesenchymal cells by recapitulating the entire sequence of events occurring during endochondral ossification. Bone morphogenetic proteins can also increase the differentiation of committed cells to the osteoblast lineage, with the subsequent formation of bone nodules and expression of markers associated with a mature osteoblast phenotype.

Bone is a highly vascularized tissue that relies on blood vessels for the transport of essential nutrients and oxygen, as well as the delivery of circulating osteogenic factors and stem cells. During fracture healing, new blood vessels sprout from existing blood vessels to restore blood supply and to facilitate bone regeneration. Inadequate bone vascularity is associated with decreased bone formation and bone mass. Direct injection of angiogenic proteins requires delivery of supra physiologic concentrations for a therapeutic effect owing to the protein's short half-life (in the order of minutes). In addition, injection of excessive concentrations of angiogenic factors may cause undesirable or potentially dangerous side effects, such as leaky blood vessels or hemorrhage. Localized vascular endothelial growth factor delivery has proven beneficial for bone regeneration in numerous animal models by promoting neovascularization, bone turnover, osteoblast migration and mineralization.

One approach in bone tissue engineering includes the use of injectable gels. Recently, various injectable scaffolds for bone regeneration have been studied including: inorganic materials, e.g., calcium phosphate, bioactive glass; synthetic polymers, e.g., poly(ethylene glycol), poly(propylene fumarate), and natural polymers, e.g., gelatin, hyaluronic acid, fibrin, collagen, alginate and chitosan. Most of these materials are gel based injectable biomaterials whose use includes difficulties that generally revolve around issues of biocompatibility, biodegradation, toxicity, in-situ hardening to obtain conformal filling, mechanical integrity, and the difficulty in delivering drugs supporting the healing processes. In addition, many types of cells perform poorly when suspended within a gel.

Another approach in bone tissue engineering is the use of injectable microparticles. The microparticles have been studied using different materials, including chitosan, as delivery vehicles for drugs, proteins and genes. Recently, microparticles were investigated as injectable scaffolds for cartilage tissue regeneration and bone regeneration. The microparticles can be seeded with autologous cells before implantation to function as cell carriers. Microparticles were also designed to enhance host cell migration, attachment, proliferation, and differentiation once implanted. One advantage of this approach, compared with the traditional block scaffolds, is that small particles can be combined with a vehicle and can be administered by injection. The microparticles provide the possibility of filling defects of different shapes and sizes through minimally invasive surgery. Minimally invasive surgeries limit the pain, limit prolonged hospitalization, reduce recovery time, reduce blood loss, and reduce scar formation as compared with conventional open surgeries, which require implanting 3D conventional scaffolds. Upon implantation, the microparticles' vehicle system conforms to the irregular implant site, whereas the interstices between the particles may provide a void space for both tissue and vascular ingrowth that is required for effective healing. Spherical shaped bone substitutes have the flexibility in filling different geometric cavities with closer packing than nonspherical shaped substitutes. Therefore, microparticle bone substitutes have the capacity to fill irregular-shaped bone defects easily.

However, none of the above injectable microparticles is designed to integrate multiple components that will lead to osteoblast growth and function. Specifically, there are currently no defined design criteria for microparticles with spatial and temporal control over the delivery of multiple mitogenic growth factors or bone morphogenic proteins. Rather, some of the fabrication methods of microparticles use very high pressure and temperatures, and it is therefore impossible to incorporate any mitogenic growth factors or bone morphogenetic proteins into the microparticles.

The present invention provides an improved system for developing bone substitute materials that can apply for bone healing without any of the above described problems.

### SUMMARY OF THE INVENTION

On a broad aspect, there is provided herein a biomimetic microparticle that comprises one or more scaffold materials at least partially crosslinked with one or more non-toxic cross-linking agents to form a biodegradable tissue replacement/repair matrix. One or more reinforcement materials is at least partially incorporated into the biodegradable tissue replacement/repair matrix.

In certain embodiments, one or more bioactive materials are at least partially incorporated into the biomimetic microparticle.

In another broad aspect, there is provided herein a method for preparing microparticles comprising cross-linking one or more scaffold materials with one or more non-toxic cross-linking materials in an environment that avoids at least one or more conditions of: high temperatures, high pressures, high voltages, and highly toxic chemicals.

The cross-linked scaffold material at least partially forms a biodegradable tissue replacement/repair matrix. As such, the method further includes incorporating at least one or more reinforcement materials into the biodegradable tissue replacement/repair matrix. The reinforcement material at least partially neutralizes one or more acidic by-products formed during degradation of the biomimetic microparticle in situ.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1 is a scanning electron microscope (SEM) image of biomimetic microparticles.

FIG. 2 is a SEM image of a bone-like mineral layer grown on chitosan/calcium phosphate (CS/CaP) where the microparticles were incubated in 5× simulated body fluids (SBF) for 48 h.

FIG. 3 shows FTIR spectra of chitosan stock, chitosan microparticles (CS Microparticles), chitosan/calcium phosphate microparticles (CS/CaP Microparticles), and mineralized chitosan/calcium phosphate microparticles (CS/CaP Microparticles) incubated in 5× simulated body fluids (SBF) for 48 h.

FIG. 4 shows the XRD of chitosan stock, chitosan/calcium phosphate microparticles (CS/CaP Microparticles), and mineralized chitosan/calcium phosphate microparticles (CS/CaP Microparticles) (baseline was shifted).

FIG. 5A is a graph showing the temporally controlled release of vascular endothelial growth factor (VEGF encapsulated chitosan and chitosan/calcium phosphate microparticles) (CS/CaP Microparticles).

FIG. 5B is a graph showing the temporally controlled release of growth factors—IGF-1 release from bone-like mineral layer (BML) in the surfaces of chitosan/calcium phosphate microparticles (CS/CaP Microparticles).

FIGS. 6A and 6B are schematic illustrations of biomimetic microparticles showing a spatially and temporally controlled release of different growth factors upon dissolving the first mineral layer (FIG. 6A, and then microparticles FIG. 6B).

FIG. 7 is a graph showing BMSC attachment on CS Microparticles, CS/CaP Microparticles, and polystyrene wells (without MPs) at different times.

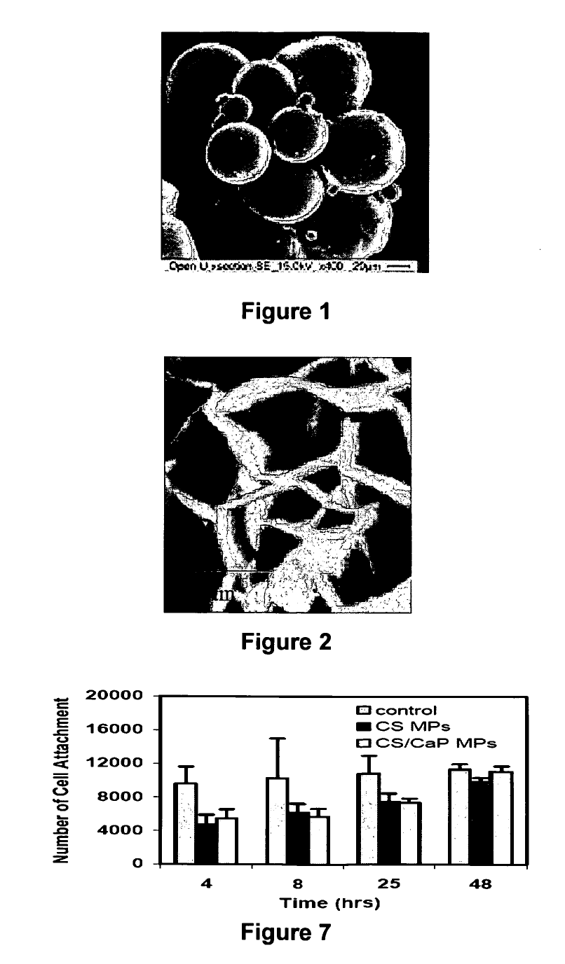

### DETAILED DESCRIPTION OF THE PREFERRED EMBODIMENT(S)

In one aspect, the present invention relates to a novel microparticle system that integrates such parameters such as osteoconductivity, osteoinductivity, and structural and mechanical integrity into a novel biomimetic microparticle, which, in turn, can lead to osteoblast growth and function towards bone regeneration.

The present system described herein also provides novel biomimetic microparticles. The biomimetic microparticles emulate the structure and mechanisms of the biological bone tissue, without the shortcomings of current artificial bone-replacement materials. The biomimetic microparticles can be prepared in a physiologically amenable environment, thereby avoiding high temperatures, high pressures, high voltages, and highly toxic chemicals.

The system described herein allows for the incorporation of more than one regenerative factors within the biomimetic microparticle, while retaining a desired bioactivity. The system provides that capability to deliver multiple osteogenic factors in a spatially and temporally controlled manner, while still maintaining the bioactivity of such factors.

In certain embodiments, the biomimetic microparticles can be combined with a suitable vehicle and can be administered by injection to a subject in need thereof.

In another aspect, the biomimetic microparticles are useful to fill defects (for example, different shapes and/or sizes) in bone tissue through minimally invasive surgery; for example, delivery of the biomimetic microparticles percutaneously (as compared with prior methods which relied on inserting 3D scaffolds into the subject).

In certain embodiments, the biomimetic microparticles can be injected with different combinations of therapeutic materials: e.g., different drugs/growth factors, a mixture of different cell types (osteoblasts and endothelial cells, osteoblasts and chondrocytes).

In one embodiment, the microparticle system can be used in a variety of applications in the bone tissue regeneration including nonunions, segmental defects, osteotomies, arthrodesis, complicated fractures, spine fusion, and dental applications.

In other embodiments, different combinations of microparticles can be used as a mixture to correct bone defects and for other therapeutic applications.

In one particular aspect, there is provided herein biomimetic microparticles that are useful for the generation of tissue in situ. The biomimetic microparticles include one or more at least partially cross-linked scaffold materials. In one embodiment, the scaffold material comprises at least one or more natural biopolymers. The scaffold material can be a linear polysaccharide, including, but not limited to at least one or more chitosan-type materials. In a particular embodiment, the scaffold material comprises about from about 10% to about 90% of the weight of the microparticle.

In certain embodiments, the scaffold material is at least partially cross-linked with at least one or more non-toxic multi-valent ionic cross-linking agents. The cross-linking agent allows the scaffold material to be at least partially cross-linked without requiring the biomimetic material being formed to be exposed to an environment having one or more potentially damaging conditions, such as, for example, high temperatures, high pressures, high voltages, and toxic chemicals. The cross-linking agent substantially prevents the biomimetic microparticle from degrading too quickly. This slow degradation of the biomimetic particle thus provides a desirable slow-release characteristic to the biomimetic microparticle. In one particular embodiment, the cross-linking agent at least partially comprises a non-toxic multi-valent material. One non-limiting example of a non-toxic multi-valent material is tripolyphosphate.

In a particular aspect, the biomimetic material is formulated such that the scaffold material is degraded once in situ. In certain embodiments, the biomimetic microparticle includes at least one or more reinforcement materials that are incorporated into a biodegradable tissue replacement/repair matrix formed by the cross-linked scaffold material. The reinforcement material provides strength to the biomimetic microparticles. In certain embodiments, one non-limiting example of a suitable reinforcement material can be an osteoconductive material. The presence of the osteoconductive reinforcement material can neutralize one or more acidic by-products that are formed during degradation of the biomimetic microparticle in situ. In certain embodiments, one non-limiting example of a suitable reinforcement material can be calcium phosphate. In other embodiments, the reinforcement material can be a calcium-containing substance, including such non-limiting examples as calcium carbonate, calcium sulfate and calcium oxide.

In another aspect, the biomimetic microparticles can further be formed with a partial coating of a bone-like mineral layer. In one non-limiting example, the coating can be a carbonated apatite mineral material.

In yet another aspect, the biomimetic microparticle can have at least one or more bioactive materials at least partially incorporated into the biomimetic microparticle. In a particular embodiment, the cross-linked scaffold material forms a biodegradable tissue replacement/repair matrix that is capable of at least temporarily encapsulating one or more bioactive materials within the scaffold material. In certain embodiments, the bioactive material can be a first bioactive material that is at least partially encapsulated in the biodegradable tissue replacement/repair matrix.

By “encapsulation”, it is meant stable association with the scaffold material and/or matrix. Thus, it is not necessary for the scaffold material and/or matrix to completely surround the bioactive material(s) as long as the material(s) is/are stably associated with the scaffold material and/or matrix when administered in vivo. Thus, “stably associated with” and “encapsulated in” or “encapsulated with” or “co-encapsulated in or with” are intended to be synonymous terms. The stable association may be achieved by a variety of means, including covalent bonding (for example, with a cleavable linkage scaffold material and/or matrix, noncovalent bonding, and trapping the bioactive material(s) in the interior of the scaffold material and/or matrix and the like). It is desired that the association be sufficiently stable so that the bioactive material(s) remain associated with the scaffold material and/or matrix at a non-antagonistic ratio until it is delivered to the target site in the treated subject.

In certain embodiments, the encapsulated bioactive material comprises at least one or more bone morphogenic factors. Also, in certain embodiments, the encapsulated bioactive material can aid in formation of at least one of chondrocytes and osteoblasts in situ during a later phase of tissue repair.

In still another aspect, the biomimetic system described herein can include at least a second bioactive material that at least partially coats a biodegradable tissue replacement/repair matrix formed by the cross-linked scaffold material. In one non-limiting example, the coating bioactive material can be at least one or more mitogenic growth factors. In certain non-limiting examples, the mitogenic growth factors can be at least one or more of transforming growth factor (TGF-beta), platelet derived growth factor (PDGF), fibroblast growth factor (FGF) and insulin-like growth factor (IGF-1).

Also, in certain embodiments, the coating bioactive material can aid in formation of soft callus in situ during at least an early phase of hard tissue repair.

Further, in certain embodiments, the biomimetic microparticle can include one or more bioactive materials that are present in an excess amount as compared to any amount of naturally occurring bioactive materials in the subject's tissue.

In one example, the first bioactive material can comprise cellular materials, including, but not limited to a patient's own cellular materials, and/or stem cells. Also, the generation of tissue can include the new growth of tissue and/or the regeneration of tissue.

In certain examples, at least one or more biomimetic microparticles can have diameters that range from about 10 microns to about 100 microns in size. At least some of the biomimetic microparticles can have a generally spherical shape.

In a further aspect, there is provided herein a tissue repair composition comprising a plurality of biomimetic microparticles. In certain embodiments, the tissue repair composition can be formulated to be readily injectable into a subject.

In still another aspect, the biomimetic microparticles can be formulated to include further desirable ingredients including, but not limited to at least one or more of glucosamine, vitamins, antimicrobial and antibiotic materials.

In a further aspect, there is provided herein an osteoimplant material that includes at least some of the biomimetic microparticles, as described herein. Also provided herein is a graft material that includes at least the biomimetic microparticles as described herein. The graft material can be at least one or more of a bone, cartilage or dental tissue.

In yet another broad aspect, there is provided herein a method for preparing biomimetic microparticles by cross-linking one or more scaffold materials. The method includes using at least one or more non-toxic cross-linking materials to form a biodegradable tissue replacement/repair matrix. The scaffold materials are at least partially cross-linked with the non-toxic cross-linking agents where the formulation environment generally avoids at least one or more potentially damaging conditions, such as high temperatures, high pressures, high voltages, and highly toxic chemicals.

In certain embodiments, the cross-linking material and the scaffold material of the biomimetic microparticle are at least partially covalently cross-linked, but are not reactive with respect to each other.

In certain embodiments, the biomimetic microparticle includes a cross-linked scaffold material that forms a biodegradable tissue replacement/repair matrix where a reinforcement material is incorporated into the biodegradable tissue replacement/repair matrix.

In another particular embodiment, the biomimetic microparticle further includes at least one or more bioactive materials that are incorporated into the biodegradable tissue replacement/repair matrix. In one non-limiting embodiment, the biomimetic microparticle includes one or more bioactive materials that are incorporated by being encapsulated in the biodegradable tissue replacement/repair matrix.

In another particular embodiment, the method further includes forming at least a partial coating of a bone-like mineral layer on the biodegradable tissue replacement/repair matrix. Also, in certain embodiments, the coating can further include at least or more bioactive materials.

The method can further include incorporating one or more types of cells into the biodegradable tissue replacement/repair matrix. The cells incorporated into the biodegradable tissue replacement/repair matrix can form a cell-to-cell adhesion matrix within the subject. In certain embodiments, the cell-to-cell adhesion matrix provides an especially desired flexibility and strength that is beneficial to the area in the subject being treated.

In still a further aspect, there is provided herein a method for forming a tissue repair composition that includes preparing a plurality of biomimetic microparticles as described herein. Such tissue repair composition can be delivered to the subject in a manner that provides the desired beneficial effect. In one embodiment, the tissue repair composition is used to generate/regenerate tissues in situ at the desired treatment area in the subject. The method for generating or regenerating tissue in situ can include injecting a desired quantity of the biomimetic microparticles. Non-limiting examples of tissue repair compositions can be used to generate/regenerate bone, cartilage, bone tissue, and the like.

Also provided herein are methods for forming an osteoimplant and/or a bone graft. The generation of hard tissue includes new growth of tissue and regeneration of tissue. Non-limiting examples of hard tissue include, but are not limited to bone, cartilage or dental tissue. The method can also include application of the biomimetic microparticles to a hard tissue defect site to promote new growth in situ.

In yet another aspect, there is provided herein a method for introducing cross-linked biomimetic microparticles into a body of a mammalian patient, comprising: (a) forming a biomimetic microparticle according to any of the preceding claims; (b) placing at least one or more of the biomimetic microparticles prepared in step (a) into the body of the patient; and, (c) allowing at least one or more of the biomimetic microparticles to degrade in situ. In certain embodiments, the step (b) can be carried out by injection. The injection can be substantially directly into the hard tissue. In certain embodiments, injection can be made at a hard tissue site in need of tissue generation.

In still a further aspect, there is provided herein a method for providing a biomimetic microparticle material that is formulated to be used as a coating on the surface of a preformed synthetic implant. The method can include: (a) forming a biomimetic microparticle as described herein; and (b) at least partially coating a preformed synthetic implant with the biomimetic microparticle prepared in step (a). In certain non-limiting methods, the step (b) can be carried out by brushing, painting, extrusion, or dipping.

In still another embodiment, there is provided a method for administering one or more types of biomimetic microparticles where each type can contain different bioactive materials. In one embodiment, the biomimetic microparticles can be applied in a layer-by-layer fashion in order to obtain a specific spatial delivery to the site under repair or regeneration. The clinician can best determine the composition of each layer and the time of delivery of each type of biomimetic microparticle to best meet the patient's needs.

The following examples are intended to illustrate preferred embodiments of the invention and should not be interpreted to limit the scope of the invention as defined in the claims, unless so specified.

### EXAMPLES

The data collected provides strong evidence of the usefulness of the microparticle system. This data includes: (i) the design and fabrication of the biomimetic microparticles themselves, (ii) the characterization of the biomimetic microparticles using analytical methods, (iii) the controlled release of growth factors from the biomimetic microparticles, (iv) determination of any degradation of the biomimetic microparticles, and (v) cultures of murine bone marrow stromal cells (BMSCs) on and/or encapsulated in the microparticles.

### Example of Method for Making Biomimetic Microparticles

In order to apply microparticles for bone regeneration, the materials used in making the biomimetic microparticles are carefully selected in order to obtain multi-functionality of the biomimetic microparticles.

In one embodiment, a chitosan-type material (“chitosan”) is a useful scaffold material. As used herein, chitosan can be generally described as a deacetylated derivative of chitin, a high molecular weight and second most abundant natural biopolymer commonly found in the shells of marine crustaceans and cell walls of fungi. Chitosan is a linear polysaccharide, composed of glucosamine and N-acetyl glucosamine linked in a β(1-4) manner; the glucosamine/N-acetyl glucosamine ratio being referred to as the degree of deacetylation. In the examples presented herein, the chitosan comprised shrimp shell chitosan (85% deacetylated) which was purchased from Sigma Chemical Co. (Milwaukee).

The presence of chitosan allows the biomimetic microparticles to be additionally useful in such further medical applications as drug delivery in both systemic and local use, and in wound dressings. The presence of chitosan can also aid in the differentiation of osteoprogenitor cells and bone formation. Also, the presence of chitosan can provide a beneficial antibacterial activity to the site being treated in the subject. In certain embodiments, the use of chitosan is especially beneficial since, in certain end-use applications, chitosan evokes a minimal foreign body reaction within the subject, with little or no fibrous encapsulation occurring. Biodegradability and biocompatibility are also desirable properties that make chitosan useful material for bone regeneration.

It is to be understood that the biomimetic microparticles described herein have improved the structural integrity and mechanical properties over those of chitosan alone.

In a particular aspect, there is provided herein a method for making biomimetic, microparticles that includes cross-linking the scaffold material using a non-toxic multivalent ionic cross-linking agent where the cross-linking agent comprises tripolyphosphate. In certain embodiments, the tripolyphosphate cross-linking agent is especially useful as a cross-linking agent for biomimetic microparticles that also include chitosan. In such embodiments, tripolyphosphate does not act as a toxic chemical cross-linker.

Also, in certain embodiments, the cross-linked biodegradable tissue replacement/repair matrix can include the incorporation of calcium phosphate into at least the biodegradable tissue replacement/repair matrix of the biomimetic microparticles. The calcium phosphate materials provide the biomimetic microparticles with enhanced mechanical properties. Also, the calcium phosphate can neutralize the acidic by-products of any polymer implants upon degradation.

Coating of Bone-Like Mineral Layer on Biomimetic Microparticles

For adequate bone tissue regeneration, there should be a bonding of the biomaterials to a living bone. Thus, in a further aspect, the biomimetic microparticles can include at least one or more mineral materials, such as bone-like mineral layer (BML) materials at least partially coated onto the exterior surface of the biodegradable tissue replacement/repair matrix biomimetic microparticles. The calcium phosphate and bone-like mineral layer show good biocompatibility and biodegradability, and are extremely osteoconductive.

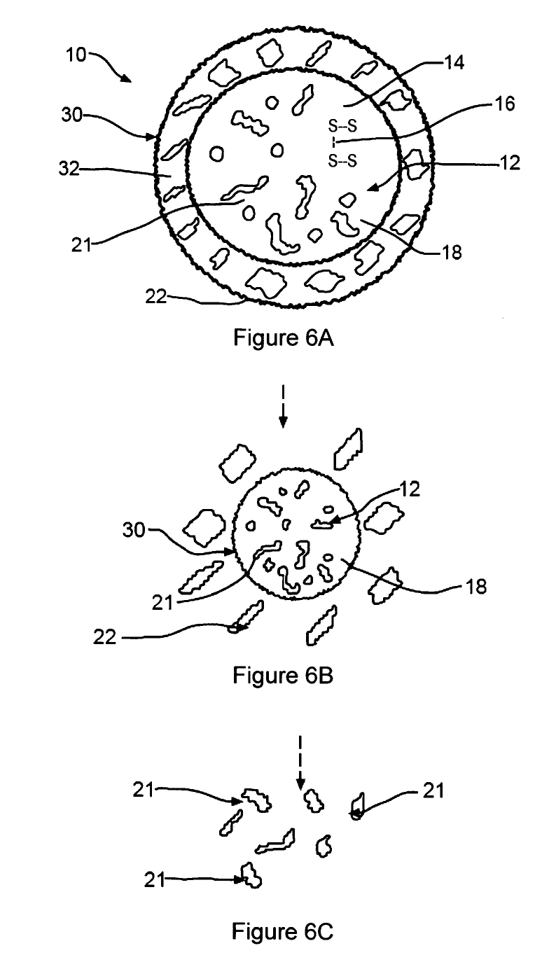

In addition, in certain embodiments, as further explained below with respect to FIGS. 6A and 6B, at least a partial coating of the bone-like mineral layer can include one or more biologically active molecules. The organic-inorganic biodegradable tissue replacement/repair matrix can be used as a carrier for bioactive molecules such as protein, drugs or growth factors, as further described herein.

TABLE 1
Ion Concentrations (in mM) of Blood Plasma and Simulated Body Fluids (SBF)

| | Na⁺ | K⁺ | Mg²⁺ | Ca²⁺ | Cl⁻ | HCO₃⁻ | H₂PO₄⁻ | SO₄²— | pH |
|---|---|---|---|---|---|---|---|---|---|
| Blood plasma | 142.0 | 5.0 | 1.5 | 2.5 | 103.0 | 27.0 | 1.0 | 0.5 | 7.2-7.4 |
| 1 × SBF | 141.0 | 5.0 | 1.5 | 2.5 | 152.0 | 4.2 | 1.0 | 0.5 | 7.4 |
| 5 × SBF | 705.0 | 25.0 | 7.5 | 12.5 | 760 | 21.0 | 5.0 | 2.5 | 6.8 |

The biomimetic microparticles are also useful to encourage the nucleation and/or growth of a continuous, bone-like carbonated apatite mineral layer; that is; a bone-like mineral layer in the biomimetic microparticles via a one-step, room temperature process.

In one embodiment, the bone-like mineral layers were coated on the surfaces of polymer scaffolds and films and were incubated in simulated body fluid at 37° C. Scanning electron microscope (SEM) images demonstrates the growth of a continuous mineral layer on the surface of Microparticles (see FIG. 2).

Also IGF-1 was co-precipitated successfully into a mineral layer by adding it into 5× simulated body fluid.

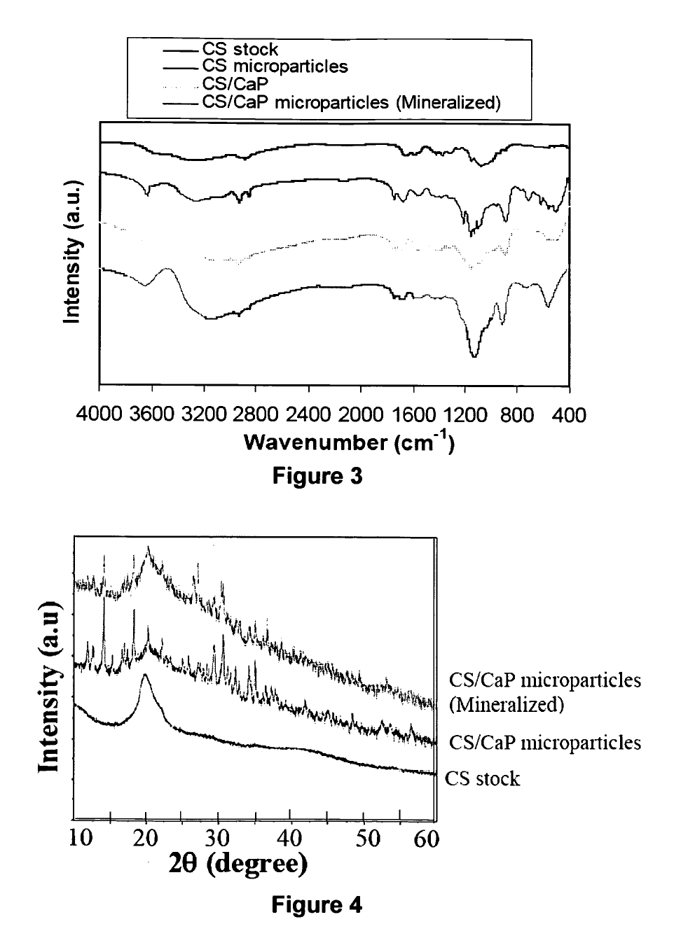

The chemical and physical structure of microparticles, Fourier transform infra-red (FTIR) and x-ray diffraction (XRD) were examined. FIG. 3 shows FTIR spectra of chitosan stock, chitosan microparticles (CS Microparticles), chitosan/calcium phosphate microparticles (CS/CaP Microparticles), and mineralized chitosan/calcium phosphate microparticles (CS/CaP Microparticles) incubated in 5× simulated body fluids for 48 h.

FIG. 4 shows the XRD of chitosan stock, chitosan/calcium phosphate microparticles (CS/CaP microparticles), and mineralized chitosan/calcium phosphate microparticles (CS/CaP microparticles) (baseline was shifted).

The IR spectra exhibit the as-received chitosan not cross-linked with tripolyphosphate, chitosan microparticles cross-linked with tripolyphosphate, and chitosan/calcium phosphate microparticles cross-linked with tripolyphosphate. For the chitosan sample, Amide I and Amide II bands appeared at 1648 and 1580 cm⁻¹, respectively. These amide bands were shifted to 1668 and 1542 cm⁻¹ after cross-linking chitosan with tripolyphosphate. The cross-linked chitosan also showed new peaks at 1741 and 1154 cm⁻¹ due to the linkage between tripolyphosphate groups and ammonium ions in the chitosan.

In addition, the characteristic phosphate bands at 1037 and 563 cm¹ appeared for both types of microparticles, confirming the presence of phosphate groups. The chitosan stock sample exhibited the main peak at 2θ=20° in XRD. This peak was observed in the same region of XRD due to the existence of chitosan in the chitosan calcium phosphate biomimetic microparticles (see FIG. 4). This XRD pattern also exhibited sharp characteristic peaks, confirming the presence of tripolyphosphate and calcium phosphate in the microparticles.

These results demonstrated that microparticles have the characteristics of both chitosan and calcium phosphate. The results also reveal the formation of cross-links between chitosan and TPP. The microparticles provide osteoconduction and unique properties of chitosan which are necessary for bone regeneration. The cross-linked microparticles also provide spherical shape and structural integrity, which is an important parameter since the microparticles can be exposed to the physiological environment for several weeks until bone tissue is formed. The spherical shape of microparticles also provides an advantage that allows the microparticles to easily fill the irregularly shaped bone defects.

Temporally Controlled Release of Growth Factors

For a VEGF release experiment, 200 mg of chitosan microparticles encapsulated VEGF (500 pg/ml) was kept in a beaker with 50 ml phosphate buffered saline (PBS) with lysozyme (18 μg/mL). The beaker was incubated at 37° C. with slight shaking. 2 ml of sample was drawn every 3 days for a period of 3 weeks and frozen. The media was replaced at each time point. ELISA was performed on the samples to determine the amount of VEGF released at different times. The same experiment was performed for microparticles (CaP/CS) encapsulated VEGF.

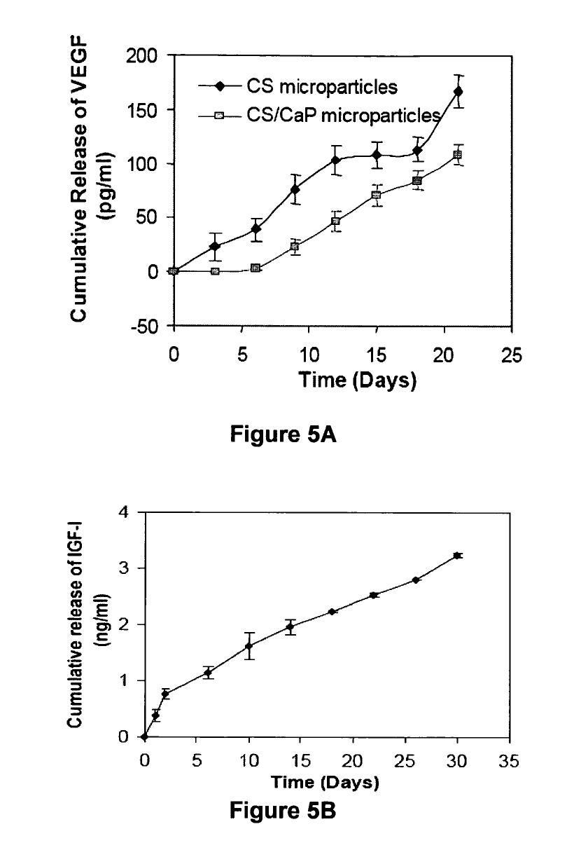

FIG. 5A shows the controlled release of VEGF from both types of microparticles. Both types of microparticles did not show the initial burst release. Chitosan/calcium phosphate microparticles exhibit more slow release of VEGF relative to chitosan microparticles due to the reinforcement effect of CaP. In order to study the growth factor release from BML, BML containing IGF-1 was coated on chitosan/calcium phosphate biomimetic microparticles. Then, mineralized microparticles were incubated in PBS at 37° C. for 14 days and determined the release of IGF-1 using ELISA. Controlled release of IGF-1 from BML was observed over time and burst release was not observed (see FIG. 5B).

Determination of any Degradation of the Biomimetic Microparticles

Before using the microparticles to study release profiles of bone morphogenic proteins, their biodegradation behavior was studied in the physiological environment. Lysozyme is the primary enzyme responsible for in vivo degradation of chitosan through hydrolysis of acetylated residues. The degradation of chitosan is related to the molecular weight and deacetylation. Highly deacetylated forms degrade after several months in vivo and produce chitosan oligosaccharides with variable length.

An enzymatic degradation solution was prepared by adding to a PBS solution (pH 7.4) a final lysozyme concentration of 18 μg/mL. This lysozyme concentration was selected in order to be compatible with a lysozyme concentration in human serum which is 9-17 μg/ml. The microparticles (50 mg) were immersed in 2 ml of the enzymatic degradation solution in sealed vials, and incubated at 37° C. up to 5 weeks without changing media. At each time point (every 4 days), the microparticles were removed from the incubation medium and washed thoroughly with deionized water, and subsequently dried.

The weight loss analysis suggested that there was no significant weight loss during the 5 weeks incubation in the enzymatic degradation solution for both types of CS microparticles. SEM results also did not show the significant degraded features for microparticles similar to weight loss data. No drastic degradation has taken place for the microparticles over a period of 5 weeks incubation in the enzymatic degradation solution, suggesting that the microparticles prepared in the above method serve excellent scaffolds in bone tissue engineering as the bone tissue takes a few weeks to develop.

In Vitro Biomimetic Microparticles

C57BL/6 murine (BMSCs) were isolated by flushing the mice femur and tibia with Dulbecco's modified Eagle's medium (DMEM) (GIBCO-BRL, Life Technologies), with 2% fetal bovine serum (FBS). The cultures were incubated at 37° C. in humidified 5% CO₂/95% air atmosphere in medium (alpha-minimum essential medium (MEM) supplemented with 10% fetal bovine serum (FBS), 100 U/ml penicillin, 100 μg/ml streptomycin sulfate). After the first passage of cell growth, the adherent cells were harvested as follows: cells were washed twice with PBS, detached from the surface by application of trypsin/EDTA for 3-5 min. at room temperature, and washed with the growth medium.

BMSCs attachment was tested at different time points after washing unattached cells twice with PBS, and then the attached cells were trypsinized and counted using a hemacytometer. BMSCs attachment on MPs at 4, 8, 25, and 48 hrs are shown in FIG. 7.

BMSCs were seeded on control polystyrene wells (without MPs), CS MPs (12 mg), and CS/CaP MPs (12 mg) at 2×104 cells per well in a 96-well plate containing standard cell medium at 37° C. The number of attached cells was increased statistically significantly at 25 hrs and 48 hrs compared with 4 and 8 hrs for both types of MPs. In addition, the number of cell attachments was not significant between control groups and MP groups at 48 hrs.

In addition, there is now evidence that continuous communication between the extracellular biodegradable tissue replacement/repair matrix and the osteoblasts is essential for their differentiation and survival. Therefore, incorporation of multiple bioactive molecules in the biomaterial design is essential for the controlled induction and maintenance of osteoblast differentiation. The following design criteria and their associated rationale were used to formulate the initial design of the microparticles (Table 2).

TABLE 2
Features and Advantages of Biomimetic Microparticles useful in bone
regeneration with controlled release of multiple growth factors

| Features of the | |
|---|---|
| Biomimetic Microparticles | Advantages |
| Biocompatibility | Provide appropriate host response minimizing |
| | immunologic response |
| Osteoconductivity | Provide an appropriate environment for |
| | attachment, growth and function of osteoblast |
| Osteoinductivity | Induce mesenchymal stem cells (hMSCs) to |
| | differentiate into osteoblast, thereby |
| | introducing multiple growth factors |
| Improve mechanical properties | Support loads encountered at the implant site |
| Cross-links between scaffold material (e.g., | Improve structural integrity of tissue, |
| chitosan) and cross-linking agent (e.g., | and |
| tripolyphosphate) | Control over release rates of growth actors |
| Biodegradability | Degrade into non-toxic molecular species that |
| | are easily metabolized or excreted avoiding |
| | surgical removal |
| Tunable spatial and temporal release of | Achieve desired growth factor release profile to |
| multiple factors | enhance osteoblast differentiation and growth |
| Mild fabrication methods | Incorporate growth factors while retaining their |
| | bioactivity |
| Void spaces between the microparticles | Facilitate osteoblast migration and conduction |
| Injectable | Use minimally invasive or non-invasive |
| | methods to treat bone defects |
| In vitro cell growth on microparticles to form | Remains at the implant site without diffusing |
| microparticles/cell extracellular | throughout body |
| biodegradable tissue replacement/repair | |
| Microarchitecture | Fill irregular bone defects easily using a vehicle |
| Multiple carriers for biomimetic | Provides cell-to-cell adhesion in biodegradable |
| microparticles (e.g., blood, bone marrow, etc) | tissue replacement/repair matrix |

Incorporation of Bioactive Materials into Biomimetic Microparticles

The natural processes of bone formation and repair require the coordinated expression of many bioactive materials and/or molecules. Those skilled in the art recognize that even though high doses of growth factors can be injected in an aqueous form, it might be difficult to maintain the desired biological effects in vivo for a certain period of time, because of the short half-life of the growth factors in the body. Therefore, it is desirable to achieve delivery of growth factors in vivo in controllable manner.

In a particular embodiment, the biomimetic microparticles are formed to release different types of bioactive material in a control manner. In a particular embodiment, the biomimetic microparticles are formed so that a first type of bioactive material (such as, for example, one or more mitogenic growth factors) is released from the biomimetic microparticles earlier than a second type of bioactive material (such as, for example, bone morphogenic proteins). In a particular embodiment, at least the first bioactive material is encapsulated within a coating on the biomimetic microparticle. In one non-limiting example, the first bioactive material is formed on a bone-like mineral layer on the biomimetic microparticle.

Several non-limiting examples of useful materials that can serve as the first or second bioactive materials include mitogenic growth factors, bone morphogenic proteins, and specific transcription factors. In particular, mitogenic growth factors such as insulin-like growth factors (IGFs) and platelet-derived growth factors (PDGF) act as mitogenic growth factors. These materials can, in certain embodiments, be especially useful since these mitogenic materials are widely distributed in the soft callus of a subject early in fracture repair, while bone morphogenic proteins are associated with the chondrocytes and osteoblasts are often present later in the healing process.

Referring now to FIGS. 6A and 6B, one embodiment of a biomimetic microparticle 10 is schematically illustrated. The biomimetic microparticle 10 includes a biodegradable tissue replacement/repair matrix 12 comprising one or more scaffold materials 14 that are cross-linked by at least one or more non-toxic cross-linking materials 16. In FIG. 6A, a greatly exaggerated schematic illustration shows the scaffold material 14 as “s” and the cross-linking agent 16 as “—”, only to aid in the discussion herein.

The biodegradable tissue replacement/repair matrix 12 is formed in an environment that avoids at least one or more potentially damaging conditions, such as high temperatures, high pressures, high voltages, and highly toxic chemicals. In certain embodiments, the scaffold material 14 and the cross-linking material 16 are at least partially covalently cross-linked, but are not reactive with respect to each other.

In a particular embodiment, the biodegradable tissue replacement/repair matrix 12 further includes at least one type of an osteoconductive reinforcement material 18 that can be incorporated into the biodegradable tissue replacement/repair matrix 12.

In a particular embodiment, the cross-linked biodegradable tissue replacement/repair matrix 12 can further include at least one or more first bioactive materials 21.

In the embodiment illustrated in FIG. 6A, the biomimetic particle 10 includes the first bioactive material 21 incorporated in at least the biodegradable tissue replacement/repair matrix 12. In one non-limiting embodiment, one or more first bioactive materials 21 are encapsulated into the biodegradable tissue replacement/repair matrix 12.

In another particular embodiment, the biomimetic microparticle 10 further includes at least a partial coating 30 on the microparticle 10. In one non-limiting example, the coating material is comprised of one or more minerals or inorganic materials 32. In one embodiment, the coating 30 comprises a bone-like mineral layer (BLM) material 32 that substantially surrounds, or coats, an outer surface of the biodegradable tissue replacement/repair matrix 12. In one method for forming the biomimetic microparticle 10, the BML material 32 is layered onto the biodegradable tissue replacement/repair matrix 12 and is incubated in simulated body fluid (SBF) at substantially room temperatures.

Incorporation of Bioactive Materials into Bone-like Mineral Layer (BLM) of Biomimetic Microparticles

In another particular embodiment, bioactive materials can be incorporated into the bone-like mineral layer 32 of the biomimetic microparticle 10. In one method, the bioactive materials are co-precipitated into the bone-like mineral layer 32.

In one method, co-precipitation of bioactive molecules (such as, for example growth factors, amino acids, and peptides) into the bone-like mineral layer is achieved by a one-step biomimetic method using a simulated body fluid. Unlike in a coating method, the biological molecules interact with the crystal lattice in the bone-like mineral.

In one example, IGF-1 was co-precipitated into a mineral layer by adding IGF-1 into 5× simulated body fluid. The resulting hybrid biomimetic microparticles (30 mg) were incubated at 37° C. in 5 ml of 5× simulated body fluid (Table 1) containing 1 μg/ml of IGF-1.

Incorporation of Second Type of Materials into Biomimetic Microparticles

In another non-limiting embodiment, the coating 30 on the biomimetic microparticle 10 can further include one or more second types of bioactive materials 22. In one method for making the coating 30, the bone-like mineral material 32 and the second bioactive material 22 can be co-precipitated onto the biodegradable tissue replacement/repair matrix 12.

In certain embodiments, the formation of an organic-inorganic biodegradable tissue replacement/repair matrix 12 is achieved due to the gentle co-precipitating processing conditions that occur during the coating process. In one embodiment, the second bioactive material 22 is co-precipitated into the bone-like mineral layer material 32 by being added to a simulated body fluid at substantially room temperatures.

Formation of Hybrid Biomimetic Microparticles

In another non-limiting embodiment, hybrid biomimetic microparticles can be formed.

The shrimp shell chitosan (85% deacetylated) was purchased from Sigma Chemical Co. (Milwaukee). All common chemicals used to fabricate the biomimetic microparticles were purchased from Sigma. The chitosan solution (1.5% Or 2%, w/v) was prepared by dissolving the chitosan in dilute acetic acid (1%, v/v) at room temperature and filtering through nylon cloth to remove any insoluble component. The chitosan solution (4.5 ml) was mixed with an equal volume of acetone. The mixture (9 ml) was then emulsified into 150 ml of cotton seed oil containing 0.2% w/w Span 85 under mechanical stirring (500 rpm, Corning Stirrer) at 37° C. The system was maintained under agitation for 14 h to allow complete evaporation of the non-oil solvent. Ionic cross-linking of the chitosan in the microparticles in the oily suspension medium was achieved by addition of a tripolyphosphate solution in water (1 ml, pH 8.5±0.1) in concentrations corresponding to 32% of the amount of chitosan (w/w). After 4 h of cross-linking, the biomimetic microparticles were isolated by vacuum filtration, washed with an equal volume of n-hexane, and freeze-dried. Calcium phosphate was incorporated directly into the chitosan solution by dissolving to a final concentration of 20% (w/w of polymer) and stirred for 1 h to obtain homogeneity.

Scale-up Procedure for Microparticles

Successful scale-up of the processing parameters is important in order to obtain a desired high yield of microparticles. Successful scale-up also includes forming the biomimetic microparticles without compromising their desired characteristics.

The above described 1× batch was scaled up to 4× by mixing 25 ml of chitosan (CS) solution with equal volume of acetone. 36 ml of the mixture was then added drop-wise into 600 ml cottonseed oil mixed with 4 ml of span 85. The mixture was stirred for 14 hrs at 37° C. and an agitation speed of 870 rpm. 64% (w/v) of tripolyphosphate was mixed with 4 ml of deionized water and then added to the reaction mixture. 4 hrs after the addition of tripolyphosphate, an equal volume of hexane was added to the mixture. The resulting mixture was vacuum filtered and dried.

Release of Second Bioactive Materials

In a particular embodiment, the biomimetic microparticles are designed to release the second bioactive materials 22 (for example, mitogenic growth factors) at a point in time earlier than release of the first bioactive materials 21 (for example, bone morphogenic proteins) as schematically illustrated in FIGS. 6B and 6C.

In the embodiment illustrated in FIGS. 6A-6C, the first bioactive materials 21 are incorporated into the biodegradable tissue replacement/repair matrix 12 by encapsulation. Next, the second bioactive materials 22 are combined with the bone-like mineral material 32 and then are coated onto at least the exterior surface of the biodegradable tissue replacement/repair matrix 12. The resulting biomimetic microparticles have first and second bioactive materials 21 and 22 incorporated in two different locations. The resulting biomimetic microparticles also provide spatially located desired (e.g., osteogenic) bioactive materials within the microparticles. The biomimetic microparticles provide the clinician with the capability of spatially and/or temporally controlling the delivery of multiple beneficial (e.g., osteogenic) factors independently.

In still a further aspect, there is provided herein a method for forming a tissue repair composition that includes preparing a plurality of biomimetic microparticles as described herein. The method for generating tissue in situ can include injecting a desired quantity of the biomimetic microparticles into a subject in need thereof.

In a particular embodiment, the biomimetic microparticles are useful to “fill in” non-union areas in the bone and/or dental tissues in the subject. The non-union areas in the bone can be present as a result of, for example, disease, genetic defect, surgery trauma or tumor.

In one particular embodiment, the biomimetic microparticle compositions described herein are particularly useful in the preparation of implanted devices (such as, for example, hip replacement devices, artificial limbs, etc.) for use in a variety of medical applications.

In a general method for preparing the implanted device, a mixture of one or more biomaterial and at least one cross-linking agent is prepared as described herein.

The reaction mixture for the microparticles may be extruded into molds of various sizes and shapes, preferably before significant cross-linking has occurred between the biomaterial and the cross-linking agent (or mixture of cross-linking agents). The time will vary depending upon the type and concentration of both the biomaterial and the cross-linking agent(s) in the reaction mixture. In one non-limiting example, the time can be generally within the range of about 5 to about 60 minutes. In certain embodiments, the implant-forming microparticle reaction mixture is removed from the mold only after adequate time has elapsed to allow for equilibrium cross-linking to occur in the reaction mixture. It is also within the contemplated scope of the present invention, however, that in certain embodiments, the implant can be removed from the mold prior to the completion of the cross-linking so that the implant can be incorporated into the body of a subject.

The methods for forming the implants can include applying the reaction mixture for the microparticles (for example, by, brushing, painting, dipping or extrusion) onto one or more surfaces of a preformed synthetic implant; and allowing the cross-linking to occur in place. In certain embodiments, a cross-linked microparticle coating is formed on the surface of the implant. In other embodiments, the cross-linked microparticle compositions can be used as injectable formulations to augment the bone tissues of the subject.

In a further aspect, the biologically active agents can be incorporated into the microparticle compositions described herein. The biologically active agents can be mixed into the reaction mixture used for preparing microparticles. In another method, the biologically active agents can be covalently bound to one or more of the ingredients in the microparticle reaction mixture prior to combining such ingredients. The biologically active agents may serve to recruit cells to the area of the implant further anchoring the implant to host tissue, and may accelerate wound healing when administered to a wound site.

Non-limiting examples of useful biologically active agents include, for example, antibiotics, anti-fungals, anti-virals, anti-parasitics, cytokines, growth factors, vitamins, mineral supplements, angiogenic factors, mucopolysaccharides, cells, and other wound healing agents and the like. Other non-limiting examples can be agents for the treatment, prevention, diagnosis, cure or mitigation of disease or illness, substances which affect the structure or function of the body, or drugs. The biologically active agents can be used, for example, to facilitate implantation of the microparticles into a patient and to promote subsequent integration and healing processes.

The terms “comprising”, “consisting of”, and “consisting essentially of” are defined according to their standard meaning and may be substituted for one another throughout the instant application in order to attach the specific meaning associated with each term.

The term “biodegradable”, as used herein, means capable of being biologically decomposed. A biodegradable material differs from a non-biodegradable material in that a biodegradable material can be biologically decomposed into units which may be either removed from the biological system and/or chemically incorporated into the biological system.

By “subject” is meant an individual. Preferably, the subject is a vertebrate subject, including a mammal such as a primate, and, more preferably, a human. The term “subject” can include domesticated animals, such as cats, dogs, etc., livestock (e.g., cattle, horses, pigs, sheep, goats, etc.), laboratory animals (e.g., mouse, rabbit, rat, guinea pig, etc.), and birds, including domestic, wild and game birds such as cocks and hens including chickens, turkeys, and the like. The term does not denote a particular age. Thus, both adult and newborn subjects are intended to be covered.

“Treatment” or “treating” means to administer a composition to a subject or a system with an undesired condition or at risk for the condition. The condition can include a disease or a predisposition to a disease. The effect of the administration of the composition to the subject can have the effect of but is not limited to reducing or preventing the symptoms of the condition, a reduction in the severity of the condition, or the complete ablation of the condition. The term “treatment” as used herein denotes curative as well as prophylactic treatment.

The term “pharmaceutical” refers to biologically active compounds such as antibiotics, antiviral agents, growth factors, hormones, and the like. By “pharmaceutically acceptable” or “pharmacologically acceptable” is meant a material which is not biologically or otherwise undesirable, i.e., the material may be administered to an individual along with the microparticle formulation without causing any undesirable biological effects in the individual or interacting in a deleterious manner with any of the components of the composition in which it is contained.

The biomimetic microparticles can be administered and dosed in accordance with good medical practice, taking into account the clinical condition of the individual subject, the site and method of administration, scheduling of administration, age, sex, body weight and other factors known to medical practitioners. The choice of carrier will be determined in part by the particular active ingredient, as well as by the particular method used to administer the composition. Accordingly, there is a wide variety of suitable pharmaceutical compositions that may be suitable. Also, as described herein, the biomimetic microparticles can possess controlled release characteristics in order to provide an extended duration of effectiveness. Several terms may be used to describe various types of controlled release characteristics. For example, controlled release may refer to any modified active compound release such as delayed release, prolonged release, constant or zero-order release, extended release, sustained release, slow release, biphasic release etc.

It is to be understood that the biomimetic microparticles can be rendered injectable by suspending them in an appropriate, physiologically acceptable liquid carrier which is preferably based on water, even though other biocompatible solvents such as ethanol, glycerol, propylene glycol, polyethylene glycol, or other organic solvents may be present. In certain embodiments, the liquid constituent of the liquid carrier is aqueous and substantially free of organic solvents. In other embodiments, the incorporation of other pharmaceutical excipients may be useful or needed to optimize the properties of the formulation, such as the tolerability, the performance in terms of drug release, and the stability. This may be true for both the microparticles themselves and the liquid carrier.

Either phase may contain one or more additives which are physiologically tolerable.

It is also to be understood that, in certain embodiments, to avoid the agglomeration of the biomimetic microparticles when suspending them in an aqueous carrier, the aqueous carrier may also contain one or more physiologically acceptable surfactants. In fact, depending on the actual presentation of the dosage form, a needed excipient such as a surfactant may be incorporated either into the aqueous carrier or into a dry composition comprising the microparticles. For example, selecting an appropriate surfactant may also help to ensure that the microparticles are quickly and easily reconstituted, such as in no more than about 3 minutes, or preferably within about 60 seconds, and more preferably in no more than about 30 seconds. Examples of potentially useful surfactants include poloxamers, polysorbates, phospholipids, and vitamin E-TPGS.

In a further embodiment, there is provided a pharmaceutical kit comprising the biomimetic microparticles described herein. The pharmaceutical kit may be defined as a set of at least two compositions which are to be combined and used for a specific therapeutic, preventive, or diagnostic purpose. In one embodiment, the kit can include a first sealed compartment and a second sealed compartment which may be members of the same or of two different primary packages. The first compartment can contain the biomimetic microparticles as described herein in substantially dry form, whereas the second compartment can contain an aqueous liquid carrier for reconstituting this dry composition into an injectable microparticle suspension. Optionally, the kit can contain two or more sets of each of the first and the second compartment.

It is to be understood, that in certain kits, the substantially dry composition comprised in the first compartment resembles one single dose to be injected, and usually also the second compartment will hold the volume of liquid carrier needed to reconstitute the content of the first compartment. In other kits, the compartments may contain more than one dose to be injected at one time. The first and the second compartments may represent different chambers of a single device or a single primary package. For example, they may be the two chambers of a dual chamber syringe. The advantage of pre-filled dual chamber syringes is that the preparation and administration is safe and convenient as it does not require the handling of several containers under aseptic conditions. Alternatively, the two compartments of a set may be members of two different primary containers or packages. For example, the first compartment comprising the substantially dry biomimetic microparticle composition may be provided in the form of a sealed bottle or vial from suitable glass or plastic, and the aqueous liquid carrier may be provided in a bottle, vial, or ampoule. In a further embodiment, the first compartment is the chamber of a syringe and the second compartment is provided as a bottle, vial, or ampoule. In other kits, one of the containers can be designed as a cartridge for an auto-injecting device. Upon combining the dry composition and the aqueous liquid carrier, the ready-to-use liquid suspension is kept in the cartridge and can be loaded into the auto-injector.

It is to be further understood that either the substantially dry composition in the first compartment or the aqueous liquid carrier, or both, may comprise one or more further excipients, such as fillers, bulking agents, surfactants, preservatives, acids, bases, salts, sugars, sugar alcohols, amino acids, stabilizers, antioxidants, polymers, buffers, polyols, proteins such as human serum albumin, and plasticizers.

It is also to be understood that the dry composition (comprising the microparticles) and the aqueous liquid carrier are adapted to yield a reconstituted suspension which is suitable for injection, i.e., which is sterile, relatively isotonic and isoosmotic, and substantially free of ingredients which are toxic when administered parenterally. The viscosity should be low enough to allow injection and the capability of being administered refers to rheological properties which allow the injection with any desired delivery device.

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope.

### REFERENCES

The publication and other material used herein to illuminate the invention or provide additional details respecting the practice of the invention, are incorporated be reference herein, and for convenience are provided in the following bibliography.
1. Greenwald S A, Boden S D, Goldberg V M, Khan Y, Laurencin C T, Rosier R N. Bone-graft substitutes: facts, fictions, and applications, J Bone Joint Surg 83-A: 98-103, 2001
2. Laurencin C T, Khan Y. Bone graft substitute materials at web site for emedicine.com/orthoped/topic611.
3. Laurell L, Gottlow J, Zybutz M, Oersson R. Treatment of intrabony defects by different surgical procedures: a literature review, J Periodontol 69: 303-313, 1998.
4. Friedlander G E. Currents concepts review: bone grafts: the basic science rationale for clinical applications, J Bone Joint Surg 69A: 786-790, 1987.
5. Rees D C, Haddad F S. Bone transplantation, Hosp. Med. 64: 205-209, 2003.
6. Kohn D H, Ducheyne P. Materials for Bone, Joint and cartilage replacement, In: Medical and Dental materials, D F Williams Ed, VCH Verlagsgesellschaft, FRG, 29-109, 1992.
7. Kohn D H. Bioceramics, In: Biomedical Engineers Handbook, M Kurtz, Ed, McGraw-Hill, NY, PP. 13.1-13.24, 2002.
8. Younger E M, Chapman M W. Morbidity at bone graft donor sites, J Ortho Trauma 3:192-195, 1989.
9. Li P, Calvert P, Levy R J, Kokubo T, Schneid C R. Eds, Mineralization in natural and synthetic biomaterials, Materials Research Society, Warrendale Pa., 2000.
10. Langer R and Vacanti J P. Tissue Engineering, Science 260: 920-926, 1993.
11. Laurencin C T, El-Amin S F, Ibim S E, Willoughby D A, Attawia M, Allcock H R, Ambrosio A A. A highly porous 3-dimensional polyphosphazene polymer matrix for skeletal tissue regeneration, J of Biomed Mater Res 30: 133-138, 1996.
12. Hollinger J. Strategies for regenerating bone of the craniofacial complex, Bone 14:575-580, 1993.
13. Ishaug-Riley S L, Crane G M, Miller M J, Yasko A W, Yaszemski M J Mikos A G. Bone formation by three-dimensional stromal osteoblast culture in biodegradable polymer scaffolds. J Biomed Mater Res 36: 17-28, 1997.
14. Krebsbach P H, Kuznetsov S A, Satomura K, Emmons R V, Rowe D W, Robey P G. Bone formation in vivo: comparison of osteogenesis by transplanted mouse and human marrow stromal fibroblasts, Transplantation 63: 1059-1069, 1997.
15. Kuznetsov S A, Krebsbach P H, Satomura K, Kerr J, Riminucci M, Benayahu D, Robey P G. Single-colony derived strains of human marrow stromal fibroblasts form bone after transplantation in-vivo, J of Bone & M M Res 12: 1335-1347, 1997.
16. Krebsbach P H, Mankani M H, Satomura K, Kuznetsov S A, Robey P G. Repair of craniotomy defects using bone marrow stromal cells, Transplantation 66: 1272-1278, 1998.
17. Jayasuriya A C, Gallagher S P, Shin K, Kohn D H. Environmentally responsive micro porous PLGA scaffolds incorporating buffer salts for bone tissue engineering, (Accepted to J Biomed Mater Res).
18. Kim H D, Valentini R F. Hyaluronana-based biodegradable scaffolds for skeletal tissue reconstruction, Trans Soc for Biomat 236, 1996.
19. Payne R G, McGonigle J S, Yaszemski M J, et al, Biomat 23: 4381-4387, 2002.
20. Alsberg E, Anderson K W, Alberiuti A, et al., J Dent Res 80: 2025-2029, 2001.
21. Ducheyne P, El-Ghannam A, Shapiro I M. Effect of bioactive glass templates on osteoblast proliferation and in-vitro synthesis of bone-like tissue, J Cell Biochem 56: 162-167, 1994.
22. El-Ghannam A, Ducheyne P, Shapiro I M. Porous bioactive glass and hydroxyl apatite ceramic effect bone cell function in-vitro along different time lines, J Biomed Mater Res 36: 167-180, 1997.
23. Ohgushi H, Okumura M, Tamai S, Shors E C, Caplan A I. Marrow cell induced osteogenesis in porous hydroxyapatite and tricalcium phosphate: a comparative histomorphometric study of ectopic bone formation. J Biomed Mater Res 24: 1563-70, 1990.
24. Ohgushi H, Miyake J, Tateishi T. Mesenchymal stem cells and bioceramics: strategies to regenerate the skeleton, Novartis Foundation Symposium 249: 118-27, 2003.
25. Thomson R C, Yaszemski M J, Powers J M, Mikos A G. Investigator: McIntire L V. Hydroxyapatite fiber reinforced poly (alpha-hydroxy ester) foams for bone regeneration. Biomat 19: 1935-1943, 1998.
26. Franceschi R T. Biological approaches to bone regeneration by gene therapy. J Dent Res 84(12):1093-103, 2005.
27. Hughes F J, Turner W, Belibasakis G, Martuscelli G. Effects of growth factors and cytokines on osteoblast differentiation. Periodontol 41: 48-72, 2000.
28. Antosz M E, Bellows C G, Aubin J E. Effects of transforming growth factor B and epidermal growth factor on cell proliferation and the formation of bone nodules in isolated fetal rat calvaria cells. J Cell Physiol 140: 386-395, 1989.
29. Anusaksathien O, Webb S A, Jin Q M, Giannobile W V. Platelet-derived growth factor gene delivery stimulates ex vivo gingival repair. Tissue Eng 9: 745-756, 2003.
30. Jensen S S, Broggini N, Weibrich G, Hjorting-Hansen E, Schenk R, Buser D. Effect of Bio-Oss with or without platelet-derived growth factor on bone formation by “guided tissue regeneration”: a pilot study in rats. J Clin Periodontol 32(12):1254-60, 2005.
31. Aubin J E. Advances in the osteoblast lineage. Biochem Cell Biol 76: 899-910, 1998.
32. Gangji V, Rydziel S, Gabbitas B, Canalis E. Insulin-like growth factor II promoter expression in cultured rodent osteoblasts and adult rat bone. Endocrinology 139: 2287-2292, 1998.
33. Takayama S, Murakami S, Shimabukuro Y, Kitamura M, Okada H. Periodontal regeneration by FGF-2 (bFGF) in primate models. J Dent Res 80: 2075-2079, 2001.
34. Okuda K, Kawase T, Momose M, Murata M, Saito Y, Suzuki H, Wolff L F, Yoshie H. Platelet-rich plasma contains high levels of platelet-derived growth factor and transforming growth factor-beta and modulates the proliferation of periodontally related cells in vitro. J Periodontol 74: 849-857, 2003.
35. Masuda K, Takegami K, An H, Kumano F, Chiba K, Andersson G B, Schmid T, Thonar E. Recombinant osteogenic protein-1 upregulates extracellular matrix metabolism by rabbit annulus fibrosus and nucleus pulposus cells cultured in alginate beads. J Orthop Res 21(5): 922-930, 2003.
36. Ashton B A, Allen T D, Howlett C R, Eaglesom C C, Hattori A, Owen M, Formation of bone and cartilage by marrow stromal cells in diffusion chambers in vivo, Clin Orthop Rel Res 151: 294-307, 1980.
37. Kruyt M C, Persson C, Johansson G, Dhert W J, de Bruijn J D. Towards injectable cell-based tissue-engineered bone: the effect of different calcium phosphate microparticles and pre-culturing. Tissue Eng 12(2): 309-17, 2006.
38. Hench L L, Xynos I D, and Polak J M. Bioactive glasses for in situ tissue regeneration. J Biomater Sci Polym Ed 15: 543-562, 2004.
39. Burdick J M, Mason M N, Hinman A D, Thorne K, and Anseth K S. Delivery of osteoinductive growth factors from degradable PEG hydrogels influences osteoblast differentiation and mineralization. J Control Release 83: 53-63, 2002.
40. Payne R G, Yaszemski M J, Yasko A W, and Mikos A G. Development of an injectable, in situ crosslinkable, degradable polymeric carrier for osteogenic cell populations. Part 1. Encapsulation of marrow stromal osteoblasts in surface crosslinked gelatin microparticles. Biomaterials 23: 4359-4371, 2002.
41. Gutowska A, Jeong B, and Jasionowski M. Injectable gels for tissue engineering. Anat Rec 263: 342-349, 2001.
42. Burg K J L, Boland T, Minimally invasive tissue engineering composites and cell printing. IEEE Engineering in Medicine and Biology Magazine, pp 84-91, September/October 2003.
43. Sinha V R, Goyal V, Bhinge J R, Mittal B R, Trehan A. Diagnostic microspheres: an overview. Crit. Rev Ther Drug Carrier Syst 20(6): 431-460. 2003. Review.
44. Varde N K, Pack D W. Microspheres for controlled release drug delivery. Expert Opin Biol Ther 4(1): 35-51, 2004. Review.
45. Malafaya P B, Stappers F, Reis R L. Starch-based microspheres produced by emulsion crosslinking with a potential media dependent responsive behavior to be used as drug delivery carriers. J Mater Sci Mater Med 17(4): 371-377, 2006.
46. Frenkel P A, Chen S, That T, Shohet R V, Grayburn P A. DNA-loaded albumin microbubbles enhance ultrasound-mediated transfection in vitro. Ultrasound Med Biol 28(6): 817-822, 2002.
47. Sparnacci K, Laus M, Tondelli L, Bernardi C, Magnani L, Corticelli F, Marchisio M, Ensoli B, Castaldello A, Caputo A. Core-shell microspheres by dispersion polymerization as promising delivery systems for proteins. J Biomater Sci Polym Ed 16(12): 1557-1574, 2005.
48. Quaglia F, Ostacolo L, Nese G, De Rosa G, La Rotonda M I, Palumbo R, Maglio G. Microspheres made of poly(epsilon-caprolactone)-based amphiphilic copolymers: potential in sustained delivery of proteins. Macromol Biosci 5(10): 945-954, 2005.
49. Thissen H, Chang K Y, Tebb T A, Tsai W B, Glattauer V, Ramshaw J A, Werkmeister J A. Synthetic biodegradable microparticles for articular cartilage tissue engineering. J Biomed Mater Res A. 77(3): 590-598, 2006.
50. Mercier N R, Costantino H R, Tracy M A, Bonassar L J. Poly(lactide-co-glycolide) microspheres as a moldable scaffold for cartilage tissue engineering. Biomaterials 26(14): 1945-1952, 2005.
51. Kang S W, Jeon O, Kim B S. Poly(lactic-co-glycolic acid) microspheres as an injectable scaffold for cartilage tissue engineering. Tissue Eng 11(3-4): 438-447, 2005.
52. Xu J W, Zaporojan V, Peretti G M, Roses R E, Morse K B, Roy A K, Mesa J M, Randolph M A, Bonassar L J, Yaremchuk M J. Injectable tissue-engineered cartilage with different chondrocyte sources. Plast Reconstr Surg 113(5): 1361-1371, 2004.
53. Mercier N R, Costantino H R, Tracy M A, Bonassar L J. A novel injectable approach for cartilage formation in vivo using PLG microspheres. Ann Biomed Eng 32(3): 418-29, 2004.
54. Orienti I, Cerchiara T, Luppi B, Bigucci F, Zuccari G, Zecchi V. Influence of different chitosan salts on the release of sodium diclofenac in colon-specific delivery. Int. J. Pharm 238: 51-59, 2002.
55. Portero A, Remu~na~n-Lo pez C, Criado M T, Alonso M J. Reacetylated chitosan microspheres for controlled delivery of antimicrobial agents to the gastric mucosa. J Microencap 19: 797-809, 2002.
56. Kato Y, Onishi H, Machida Y. Application of chitin and chitosan derivatives in the pharmaceutical field. Curr Pharm Biotechnol 4(5): 303-309, 2003.
57. Conti B, Giunchedi P, Genta I, Conte U. The preparation and in vivo evaluation of the wound-healing properties of chitosan microspheres. S.T.P. Pharma Sci 10: 101-104, 2000.
58. Kong L, Ao Q, Wang A, Gong K, Wang X, Lu G, Gong Y, Zhao N, Zhang X. Preparation and Characterization of a Multilayer Biomimetic Scaffold for Bone Tissue Engineering. J Biomater App12007 Jan. 25; [Epub ahead of print].
59. Seol Y J, Lee J Y, Park Y J, Lee Y M, Young-Ku, Rhyu I C, Lee S J, Han S B, Chung C P. Chitosan sponges as tissue engineering scaffolds for bone formation. Biotechnol Lett 26(13): 1037-41, 2004.
60. Arpornmaeklong P, Suwatwirote N, Pripatnanont P, Oungbho K. Growth and differentiation of mouse osteoblasts on chitosan-collagen sponges. Int J Oral Maxillofac Surg. 2007 Jan. 11; [Epub ahead of print].
61. Di Martino A, Sittinger M, Risbud M V. Chitosan: a versatile biopolymer for orthopaedic tissue-engineering. Biomaterials 26(30): 5983-5990, 2005. Review.
62. Hoemann C D, Sun J, Legare A, McKee M D, Buschmann M D. Tissue engineering of cartilage using an injectable and adhesive chitosan-based cell-delivery vehicle. Osteoarthritis Cartilage. 13(4): 318-329, 2005.
63. Li Z, Ramay H R, Hauch K D, Xiao D, Zhang M. Chitosan-alginate hybrid scaffolds for bone tissue engineering. Biomaterials. 26(18): 3919-3928, 2005.
64. Ho M H, Wang D M, Hsieh H J, Liu H C, Hsien T Y, Lai J Y, Hou L T. Preparation and characterization of RGD-immobilized chitosan scaffolds. Biomaterials. 26(16): 3197-3206, 2005
65. Phang M Y, Ng M H, Tan K K, Aminuddin B S, Ruszymah B H, Fauziah O. Evaluation of suitable biodegradable scaffolds for engineered bone tissue. Med J. Malaysia. 59 Suppl B: 198-199, 2004.
66. LeGeros R Z, LeGeros J P. Dense hydroxyapatite. In: Hench L L, Wilson J, editors. Introduction to Bioceramics. New Jersey: World Scientific. pp 139-180, 1993.
67. LeGeros R Z. Biodegradation and bioresorption of calcium phosphate ceramics. Clin Mater 14: 65-88, 1993.
68. Hench L L. Bioceramics. J Am Ceram Soc 81: 1705-1728, 1998.
69. Durucan C, Brown P W. Low temperature formation of calcium deficient hydroxyapatite-PLA/PLGA composites. J Biomed Mater Res 51: 717-725, 2000.
70. Xu H H, Quinn J B, Takagi S, Chow L C. Synergistic reinforcement of in situ hardening calcium phosphate composite scaffold for bone tissue engineering. Biomaterials. 25(6): 1029-1037, 2004.
71. Li Z, Yubao L, Aiping Y, Xuelin P, Xuejiang W, Xiang Z. Preparation and in vitro investigation of chitosan/nano-hydroxyapatite composite used as bone substitute materials. J Mater Sci Mater Med 16(3): 213-219, 2005.
72. Jayasuriya A C, Gallagher S P, Shin K, and Kohn D H, Development of Environmentally Responsive Micro Porous poly(lactide-co-glycolide) Scaffolds for Bone Tissue Engineering, Trans. Soc for Biomat 29: 637, 2003.
73. Wang L Y, Ma G H, Su Z G. Preparation of uniform sized chitosan microspheres by membrane emulsification technique and application as a carrier of protein drug. J Control Release 106(1-2): 62-75, 2005.
74. Anal A K, Stevens W F, Remunan-Lopez C. Ionotropic cross-linked chitosan microspheres for controlled release of ampicillin. Int J Pharm 312(1-2): 166-173, 2006.
75. Bhumkar D R, Pokharkar V B. Studies on Effect of pH on Cross-linking of Chitosan With Sodium Tripolyphosphate: A Technical Note. AAPS PharmSciTech 7(2) Article 50, 2006 (http://www.aapspharmscitech.org).
76. Jayasuriya A C, Darjatmoko L, Shin K, and Kohn D H, Incorporation of Poly-1-lysine into Biomimetically Mineralized Poly(lactic-co-glycolide acid) Films, Seventh World Biomat Con, 1033, 2004.
77. Kohn D H, Shin K, Hong S I, Jayasuriya A C, Leonova E, Rossello R A, and Krebsbach P H. Self-Assembled Mineral Scaffolds as Model Systems for Biomineralization and Tissue Engineering, Proceedings of the Eighth International Conference on the Chemistry and Biology of Mineralized Tissues, W. J. Landis, and J. Sodek, Editors, University of Toronto Press, Toronto, Canada, 2004.
78. Wang X, Ma J, Wang Y, He B. Structural characterization of phosphorylated chitosan and their applications as effective additives of calcium phosphate cements. Biomaterials 22: 2247-2255, 2001.
79. Jayasuriya A C, Michels E, Ebraheim N A, Fabrication and Characterization of Hybrid Microparticles, Abstract submitted for TERMIS, Toronto, Canada, June 2007.
80. Jayasuriya A C, Deshpande S, Ebraheim N A. Degradation of Chitosan based Microparticles in Physiological Media, Abstract submitted for TERMIS, Toronto, Canada, June 2007.
81. Kinney J H, Balooch M, Marshall S J, Marshall G W Jr, Weihs T P. Atomic force microscope measurements of the hardness and elasticity of the peritubular and intertubular human dentin, J Biomech Eng 118: 133-135, 1996.
82. Marshall G W Jr, Balooch M, Gallagher R R, Gansky S A, Marshall S J. Mechanical properties of the dentinoenamel junction: AFM studies of nanohardness, elastic modulus, and fracture, J Biomed Mater Res 54: 87-95, 2001.
83. Tan S, Sherman R L Jr, Ford W T. Nanoscale compression of polymer microspheres by atomic force microscopy. Langmuir 20(17): 7015-20, 2004.
84. Web cite for aruplab.com see /guides/clt/tests/clt_a69b.j sp.
85. Liu Y, Hunziker E B, Randall N X, de Groot K, Layrolle P. Proteins incorporated into biomimetically prepared calcium phosphate coatings modulate their mechanical strength and dissolution rate, Biomat 24: 65-70, 2003.
86. Stigter M, de Groot K, Layrolle P. Incorporation of tobramycin into biomimetic hydroxyapatite coating on titanium. Biomat 23: 4143-4153, 2002.
87. Jayasuriya A C, Asaad M, Jayatissa A H, and Ebraheim N A, Dissolution Behavior of Biomimetic Minerals on 3D PLGA Scaffold. Surface and Coatings Technology 200: 6336-6339, 2006.
88. Jayasuriya A C, Shah C, Goel V, and Ebraheim N A. Characterization of Biomimetic Mineral Coated 3D PLGA Scaffolds, Proceedings of ASME International Mechanical Engineering Congress and Exposition 2006.
89. Chen F M, Wu Z F, Sun H H, Wu H, Xin S N, Wang Q T, Dong G Y, Ma Z W, Huang S, Zhang Y J, Jin Y. Release of bioactive BMP from dextran-derived microspheres: a novel delivery concept. Int J Pharm 307(1): 23-32, 2006.
90. Masuda K, Takegami K, An H, Kumano F, Chiba K, Andersson G B, Schmid T, Thonar E. Recombinant osteogenic protein-1 upregulates extracellular matrix metabolism by rabbit annulus fibrosus and nucleus pulposus cells cultured in alginate beads. J Orthop Res 21(5): 922-930, 2003.
91. Liu Y, Layrolle P, de Bruijn J, van Blitterswijk C, de Groot K. Biomimetic coprecipitation of calcium phosphate and bovine serum albumin on titanium alloy. J Biomed Mater Res. 57(3): 327-35, 2001.
92. Jayasuriya A C, Darjatmoko L, Shin K, and Kohn D H, Incorporation of Poly-1-lysine into Biomimetically Mineralized Poly(lactic-co-glycolide acid) Films, Seventh World Biomat Con, 1033, 2004.
93. Jayasuriya A C, Shah C, Ebraheim N A. Mineralized 3D Scaffolds for Bone Tissue Engineering, Abstract submitted for TERMIS, Toronto, Canada, June 2007.
94. Friedenstein A J, Gorskaja U F, Kulagina N N, Fibroblast precursors in normal and irradiated mouse hematopoietic organs, Exp Hematol 4: 267-274, 1976.
95. Ashton B A, Allen T D, Howlett C R, Eaglesom C C, Hattori A, Owen M, Formation of bone and cartilage by marrow stromal cells in diffusion chambers in vivo, Clin Orthop Rel Res 151: 294-307, 1980.
96. Beresford J N, Osteogenic stem cells and the stromal system of bone and marrow, Clin Orthop Rel Res 240: 270-280, 1989.
97. Friedenstein A J, Stromal mechanocytes of bone marrow: cloning in vitro and retransplantation in vivo, In: Immunology of bone marrow transplantation, Springer-Verlag, Berlin, 19-29, 1980.
98. Owen M, Marrow stromal cells, J Cell Sci 10: 63-76, 1988.
99. Chailakhyan R K, Gerasimov Y V, Friedenstein A J, Transfer of bone marrow microenvironment by clones of stromal mechanocytes, Bull Exper Biol Med 86: 1633-1635, 1978.
100. Bennett J H, Joyner C J, Triffitt J T, Powen M E, Adipocytic cells cultured from marrow have osteogenic potential, J Cell Sci 99: 131-139, 1991.
101. Robey and Bianco, Stem cells in tissue engineering, Nature 2002.
102. Favus M J. Primer on the Metabolic Bone Diseases and Disorders of Mineral Metabolism, Fifth Edition, Published by the American Society for Bone and Mineral Research, Washington D.C., 2003.
103. Kohn D H, Sarmadi M, Chow D L, Krebsbach P H. Effects of pH on human bone marrow stromal cells in culture: implications for tissue engineering of bone, J Biomed Mat Res, 60: 292-299, 2002.
104. Rodan G A, Noda M. Gene expression in osteoblastic cells. Crit. Rev Eukaryot Gene Expr. 1(2): 85-98, 1991. Review.
105. Nieden Nlzur, Kempka G, Ahr H J. In vitro differentiation of embryonic stem cells into mineralized osteoblasts. Differentiation 71:18-27, 2003.
106. Injectable hyaluronic acid derivative with pharmaceuticals/cells—U.S. Pat. No. 7,157,080.
107. Processes for manufacturing polymeric microspheres—U.S. Pat. No. 7,094,369.
108. Multi-layer cell encapsulation for tissue engineering—U.S. Pat. No. 6,916,640.
109. Hydrogels for orthopaedic repair—U.S. Pat. No. 7,008,635.
110. Injectable microdispersions for medical applications—U.S. Pat. No. 7,026,374.
111. Blend, cross-linkable poly(propylene fumarate) for immobilization and controlled drug delivery—U.S. Pat. No. 6,884,432.
112. Bioactive, degradable composite for tissue engineering—U.S. Pat. No. 6,328,990.
113. Biodegradable microparticles that stabilize and control the release of proteins—U.S. Pat. No. 7,060,299.
114. High drug loaded injectable microparticle compositions and methods of treating opioid drug dependence—U.S. Pat. No. 7,041,320.
115. Prolonged release microparticle preparation and production of the same—U.S. Pat. No. 5,651,990.
116. Calcium mineral-based microparticle and method for the production thereof—U.S. Pat. No. 5,648,097.

1. A biomimetic microparticle comprising
one or more scaffold materials at least partially crosslinked with one or more non-toxic cross-linking agents to form a biodegradable tissue replacement/repair matrix, and
one or more reinforcement materials at least partially incorporated into the biodegradable tissue replacement/repair matrix.

2. The biomimetic microparticle of claim 1, wherein the scaffold material is configured to be at least partially degraded once in situ.

3. The biomimetic microparticle of claim 1, wherein the scaffold material comprises at least one or more natural biopolymers.

4. The biomimetic microparticle of claim 1, wherein the scaffold material comprises at least one or more linear polysaccharides.

5. The biomimetic microparticle of claim 1, wherein the scaffold material comprises from about 10% to about 90% of the weight of the microparticle.

6. The biomimetic microparticle of claim 1, wherein the cross-linking agent at least partially comprises a multi-valent non-toxic material.

7. The biomimetic microparticle of claim 1, wherein the cross-linking agent at least partially comprises tripolyphosphate.

8. The biomimetic microparticle of claim 1, wherein the reinforcement material includes at least one or more osteoconductive reinforcement materials.

9. The biomimetic microparticle of claim 1, wherein the reinforcement material is configured to at least partially neutralize one or more acidic by-products formed during degradation of the biomimetic microparticle in situ.

10. The biomimetic microparticle of claim 1, wherein the reinforcement material comprises calcium phosphate.

11. The biomimetic microparticle of claim 1, wherein the biomimetic microparticle comprises about 10 to about 35%, by weight, reinforcement material.

12. The biomimetic microparticle of claim 1, further including at least a partial coating of a bone-like mineral layer.

13. The biomimetic microparticle of claim 12, wherein the coating comprises a carbonated apatite mineral material.

14. The biomimetic microparticle of claim 1, wherein at least one or more bioactive materials are at least partially incorporated into the biomimetic microparticle.

15. The biomimetic microparticle of claim 14, wherein at least the biodegradable tissue replacement/repair matrix is capable of at least temporarily encapsulating one or more bioactive materials.

16. The biomimetic microparticle of claim 14, wherein the bioactive material comprises at least a first bioactive material at least partially encapsulated in the biodegradable tissue replacement/repair matrix.

17. The biomimetic microparticle of claim 16, wherein the first bioactive material comprises at least one or more bone morphogenic factors.

18. The biomimetic microparticle of claim 16, wherein the first bioactive material comprises cellular material.

19. The biomimetic microparticle of claim 16, wherein the first bioactive material comprises a patient's own cellular material.

20. The biomimetic microparticle of claim 16, wherein the first bioactive material comprises stem cells.

21. The biomimetic microparticle of claim 16, wherein the first bioactive material aids in formation of at least one of chondrocytes and osteoblasts in situ during a later phase of tissue repair.

22. The biomimetic microparticle of claim 16, wherein the bioactive material comprises at least a second bioactive material at least partially coating a biodegradable tissue replacement/repair matrix formed by the cross-linked scaffold material.

23. The biomimetic microparticle of claim 22, wherein the second bioactive material comprises at least one or more mitogenic growth factors.

24. The biomimetic microparticle of claim 23, wherein the mitogenic growth factors comprise at least one or more of transforming growth factor (TGF-beta), platelet derived growth factor (PDGF), osteopontin, fibroblast growth factor (FGF) and insulin-like growth factor (IGF-1).

25. The biomimetic microparticle of claim 22, wherein the second bioactive material aids in formation of soft callus in situ during at least an early phase of tissue repair.

26. The biomimetic microparticle of claim 22, wherein the second bioactive material comprises cellular material.

27. The biomimetic microparticle of claim 22, wherein the second bioactive material comprises a patient's own cellular material.

28. The biomimetic microparticle of claim 22, wherein the second bioactive material comprises stem cells.

29. (canceled)

30. (canceled)

31. (canceled)

32. A tissue repair composition comprising a plurality of biomimetic microparticles of claim 1.

33. The composition of claim 32, wherein the composition is configured to be injectable.

34. (canceled)

35. The composition of claim 32, wherein the biomimetic microparticle further includes at least one or more of biologically active agents selected from: antibiotics, anti-fungals, anti-virals, anti-parasitics, cytokines, growth factors, vitamins, glucosamine, mineral supplements, angiogenic factors, mucopolysaccharides, and other wound healing agents and the like; agents for the treatment, prevention, diagnosis, cure or mitigation of disease or illness, substances which affect the structure or function of the body, or drugs; agents to facilitate implantation of the microparticles into a patient and to promote subsequent integration and healing processes.

36. An osteoimplant comprising at least the biomimetic microparticles of claim 1.

37. A graft comprising at least the biomimetic microparticles of claim 1.

38. The graft of claim 37, wherein the graft comprises at least one or more of a bone, cartilage or dental tissue.

39. A method for preparing microparticles of claim 1 comprising cross-linking one or more scaffold materials with one or more non-toxic cross-linking materials in an environment that avoids at least one or more conditions of: high temperatures, high pressures, high voltages, and highly toxic chemicals.

40. The method of claim 39, wherein the cross-linking material and the scaffold material are at least partially covalently cross-linked, but not reactive with respect to each other.

41. The method of claim 39, the cross-linked scaffold material is configured to be at least partially degraded.

42. (canceled)

43. (canceled)

44. (canceled)

45. The method of claim 39, wherein the cross-linked scaffold material at least partially forms a biodegradable tissue replacement/repair matrix, and the method further includes incorporating at least one or more reinforcement materials into the biodegradable tissue replacement/repair matrix.

46. (canceled)

47. (canceled)

48. (canceled)

49. The method of claim 39, wherein the cross-linked scaffold material at least partially forms a biodegradable tissue replacement/repair matrix, and the method further including adding at least one or more bioactive materials at least into or onto the microparticle.

50. The method of claim 39, wherein at least the cross-linked scaffold material at least partially forms a biodegradable tissue replacement/repair matrix, and the method further includes at least partially encapsulating one or more first bioactive materials into the biodegradable tissue replacement/repair matrix.

51. (canceled)

52. (canceled)

53. The method of claim 39, wherein at least the cross-linked scaffold material at least partially forms a biodegradable tissue replacement/repair matrix, and the method further includes forming at least a partial coating of a bone-like mineral layer on the biodegradable tissue replacement/repair matrix.

54. (canceled)

55. (canceled)

56. (canceled)

57. (canceled)

58. (canceled)

59. (canceled)

60. (canceled)

61. (canceled)

62. (canceled)

63. The method of claim 39, further including one or more cells incorporated into the biodegradable tissue replacement/repair matrix.

64. The method of claim 39, further including cells incorporated into the biodegradable tissue replacement/repair matrix, wherein the cells form a cell-to-cell adhesion matrix.

65. (canceled)

66. (canceled)

67. (canceled)

68. (canceled)

69. (canceled)

70. (canceled)

71. (canceled)

72. A method for introducing a cross-linked biomimetic microparticle into a body of a mammalian patient, comprising:
(a) forming a biomimetic microparticle according to claim 1;
(b) placing at least one or more of the biomimetic microparticles prepared in step (a) into the body of the patient; and,
(c) allowing at least one or more of the biomimetic microparticles to degrade in situ.

73. The method of clam 72, wherein step (b) is carried out by injection.

74. The method of clam 73, wherein the injection is substantially directly into the tissue.

75. The method of clam 72, wherein the injection is at a tissue site in need of tissue generation.

76. A method for providing a biomimetic material as a coating on the surface of a preformed synthetic implant, comprising:
(a) providing the biomimetic microparticle of claim 1; and
(b) at least partially coating a preformed synthetic implant with the biomimetic microparticle prepared in step (a).

77. The method of claim 76, wherein step (b) is carried out by brushing, painting, extrusion, or dipping.

78. A method to treat a disease condition in a subject which method comprises administering to a subject in need of such treatment a therapeutically effective amount of a biomimetic microparticle as described in claim 1.

79. (canceled)

80. (canceled)

81. A pharmaceutical kit comprising the biomimetic microparticles described in claim 1.

82. (canceled)

83. (canceled)

84. The pharmaceutical kit as in claim 81, wherein the first compartment contains the biomimetic microparticles substantially in dry form, and the second compartment contain a liquid carrier for reconstituting this dry composition into an injectable microparticle suspension.
